(19) United States
(12) Patent Application Publication
(10) Pub. No.: US 2004/0030351 A1
(43) Pub. Date: Feb. 12, 2004

(54) Endovascular device for reducing type I leaks from endovascularly-implanted grafts

(76) Inventor: Mark C. Goldberg, Taunton, MA (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/408,489

(22) Filed: Apr. 7, 2003

Related U.S. Application Data
(60) Provisional application No. 60/370,204, filed on Apr. 5, 2002.

(51) Int. Cl. A61M 29/00

(52) U.S. Cl. 606/192; 604/103.05

(57) ABSTRACT

An anchoring device can be used to secure a vascular rivet to the wall of a blood vessel. The anchoring device has a catheter with a rivet chamber that can be positioned at a desired location within a blood vessel. The rivet can be driven through a graft and the wall of the blood vessel by either a mechanical pusher or a blast of fluid, such as carbon dioxide gas. The rivet has an optional leg with a thermal memory that curls to secure the rivet in place when it warms to body temperature. An optional elastic rivet disk holds the graft tightly to the wall of the blood vessel during the geometric changes that occur during cardiac cycles.

### BACKGROUND OF THE INVENTION

This invention relates to the endovascular grafts, and more specifically to an endovascular device for reducing type I leaks from endovascularly-implanted grafts.

An aneurysm is a dilation or enlargement of a blood vessel that exceeds twice the normal size of the respective vessel. An aneurysm can grow with time and eventually may rupture or result in embolism of clots into the lower extremities or other organ systems, depending on the location of the aneurysm. In the event of an aneurysm rupturing, bleeding will occur and often results in death of the patient.

Aneurysms have been observed in most if not all of the named arteries in the body. A frequent location for aneurysms involves the aorta, the major blood vessel that takes origin from the heart. The most common aortic aneurysms occur in the abdomen, and are frequently referred to as AAA's. The diameter of a normal aorta is often in the 2 cm range and, due to an increasing risk of rupture, surgical intervention is generally recommended for AAA's larger than 5 cm. When an AAA exceeds 6 cm, the risk of rupture increases in a logarithmic fashion relative to its diameter. The mortality of a ruptured AAA, even with emergency surgical intervention, can be as high as 90%.

Prior to techniques popularized in the 1950's, successful management of aneurysms was rare. In 1951 however, several groups reported the successful management of abdominal aortic aneurysms using a method commonly referred to as resection and grafting. In this procedure, the intestine is displaced to the right as the abdomen is explored. The aorta is clamped above and below the aneurysm, which is then opened. Thrombus that typically lines the aneurysm wall is removed. Small vessels are over-sewn to prevent bleeding. A graft (typically of a fabric such as Dacron or expanded Teflon) is then hand-sewn to the normal-sized artery above and below the aneurysm using a polypropylene suture. The aneurysm wall is then sewn over the graft to provide coverage, protection, and homeostasis. The abdomen is then closed.

This well-accepted method provides reliable results. As with any procedure, however, complications can occur. Complications can include infection, myocardial infarction, limb loss, and even death.

Bleeding during surgery is common, and often more than 1 liter of blood loss can be observed. Such blood loss obviously increases the risk of the complications already mentioned, but particularly increases the risk of a myocardial infarction. In spite of modern blood salvage methods, many patients require nonautologus blood transfusions.

Recently, a less-invasive method has been pioneered, and it may avoid many of these complications. An endovascular method of treating aneurysms involves steering a device from within a blood vessel and positioning the device within the aneurysm. A graft is delivered so the prosthetic material bypasses and excludes the aneurysm as it is attached to normal-sized borders of the respective artery. Proper selection of a target site for both the proximal and distal attachment of the graft is critical since the artery at these sites can often be diseased with hard, plate-like scales consisting of atherosclerotic plaque. Such endovascularly-delivered grafts are often reinforced with stents or stent-like infrastructures, often with a lattice-like weave, and with barbs or other mechanical mechanisms that provide a means of attachment and thus prevent leakage or migration of the graft.

However, attaching a graft with a structure that penetrates the diseased wall has risks. Incomplete penetration into the wall can be a serious complication. In some patients, incomplete penetration may result in a gap or an incomplete seal between the graft and the blood vessel, leaving the abnormal segment of artery exposed to flow and pressure as presented by the systemic system, resulting in blood continuing to flow against the weakened wall of the aneurysm. This complication has been observed in all of the known, currently-available endovascular graft systems. Leaks in patients with an endovascularly-delivered stent graft may occur as frequently as 20-30% of the time, and can result in the continued expansion of an aneurysm, and eventually in rupture of the incompletely-treated, diseased segment of the artery. This can result in death of the patient.

Leaks in endovascularly-implanted grafts can also occur because of mechanical failure or fracture of the infrastructure used to secure the graft to the artery. Repair of such complications can be equally challenging.

Detection or diagnosis of leaks and the continuing expansion of an aneurysm is a vexing issue since the aneurysm has been considered treated, and such problems rarely occur when conventional open techniques have been used to treat an aneurysm. Even when diagnosed, repair of such leaks can be quite difficult, and on occasion may require a conventional open repair of the AAA.

Thus, a means to treat leakage or prevent migration of an endovascularly-placed graft is useful for a large population of patients being treated with this less-invasive technique and technology.

### SUMMARY OF THE INVENTION

The applicant has developed a new anchoring device for endovascular treatment of aneurysms. The device uses a vascular rivet to secure a graft to the wall of a blood vessel, reducing type I endovascular-graft leaks. A firing mechanism is used to deploy the rivet from a flexible catheter, which can be temporarily positioned within the aneurysm using an extendable mounting surface.

Preferably, the deployment catheter is arranged within a flexible sheath. The sheath can be used to position the catheter at a location in a blood vessel with the rivet adjacent to a graft. The catheter may have a guidewire channel to facilitate this placement.

Once the catheter is positioned, the mounting surface is extended to secure the device in the desired location. The mounting surface may, for example, be part of a wedge-shaped balloon that extends around opposed sides of the catheter. In such an arrangement, a balloon inflation channel may be provided on the outer surface of the catheter for inflating the balloon, causing the mounting surface to extend away from the catheter.

The vascular rivet may have a flange and at least one leg. The leg may be deformable, or may have threads. When a deformable leg is used, it may be constructed of a material that has a temperature-sensitive shape. For example, the rivet leg (or legs) is straight at room temperature but has a temperature-sensitive shape memory and a curved shape at body temperature. In some arrangements, the end of the leg of the rivet may be disposed in an aperture on a rivet disk that holds the leg straight until the rivet is deployed. An elastic rivet disk may be used to hold the graft tightly to the wall of the blood vessel during the geometric changes that occur during cardiac cycles.

The catheter includes a rivet chamber that may be arranged to deploy the rivet either out the distal end of the catheter, or through a side of the catheter. The rivet can be deployed in a variety of ways. For example, it can be deployed by applying a fluid such as carbon dioxide gas to the flange on the rivet. Alternatively, a solid pusher can be used. In either event, the leg of the rivet is propelled through the graft and the wall of the blood vessel. When using a rivet with a temperature-sensitive shape, body temperature warms the legs of the rivet once they are in place, causing the legs to curve and securing the rivet and the graft in place.

A safety system that includes a tether that is releasably connected to the rivet can also be provided to retrieve the rivet if it is not successfully deployed.

### BRIEF DESCRIPTION OF THE DRAWINGS

The invention may be better understood by referring to the accompanying drawings, in which:

FIG. 1 is a perspective view of one embodiment of the invention;

FIGS. 2-4 are cross-sectional views of the first embodiment of the invention at various stages of positioning of the catheter;

FIG. 5 is a fragmentary perspective view of an alternative embodiment of the invention;

FIG. 6 is an enlarged, cross-sectional view through lines 6-6 of FIG. 5; and

FIG. 7 is a side view of a rivet after it has been deployed by one of the apparatus seen in FIGS. 1-6.

### DETAILED DESCRIPTION

The figures show two possible arrangements for an endovascular device 10 for securing a vascular rivet, staple, or screw to the wall of a blood vessel 12.

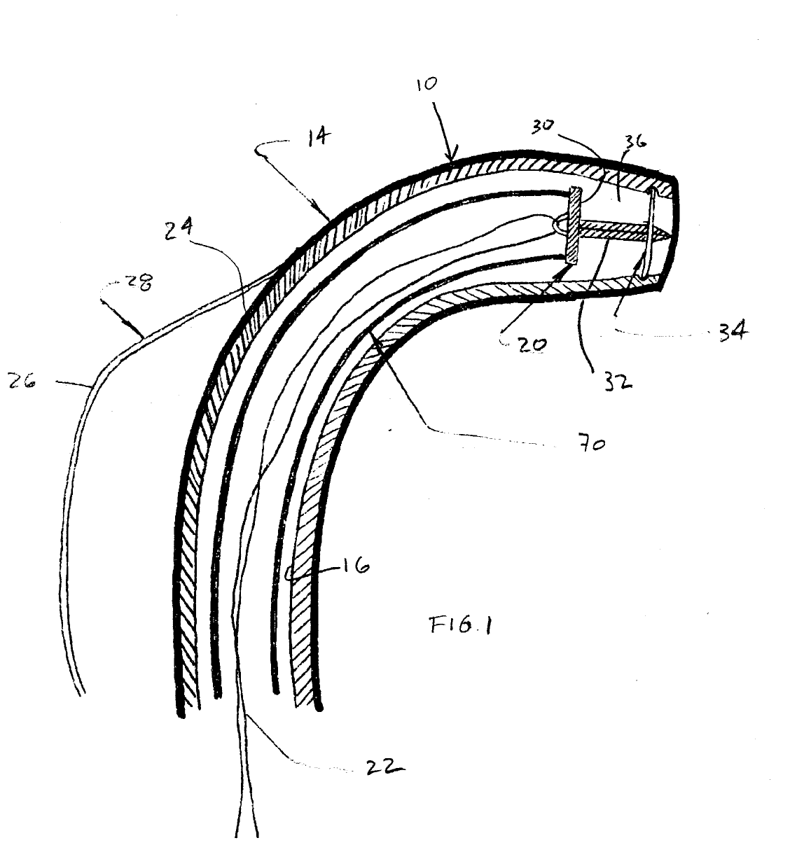

The embodiments that have been illustrated are used with a flexible sheath 13 (seen in FIGS. 2-4) that provides a passage for a deployment catheter 14. Preferably the sheath has a diameter of less than 12 French but may be as large as 24 French. The deployment catheter seen in FIG. 1 has a channel 16 with a rivet 20 that is looped with a tether 22.

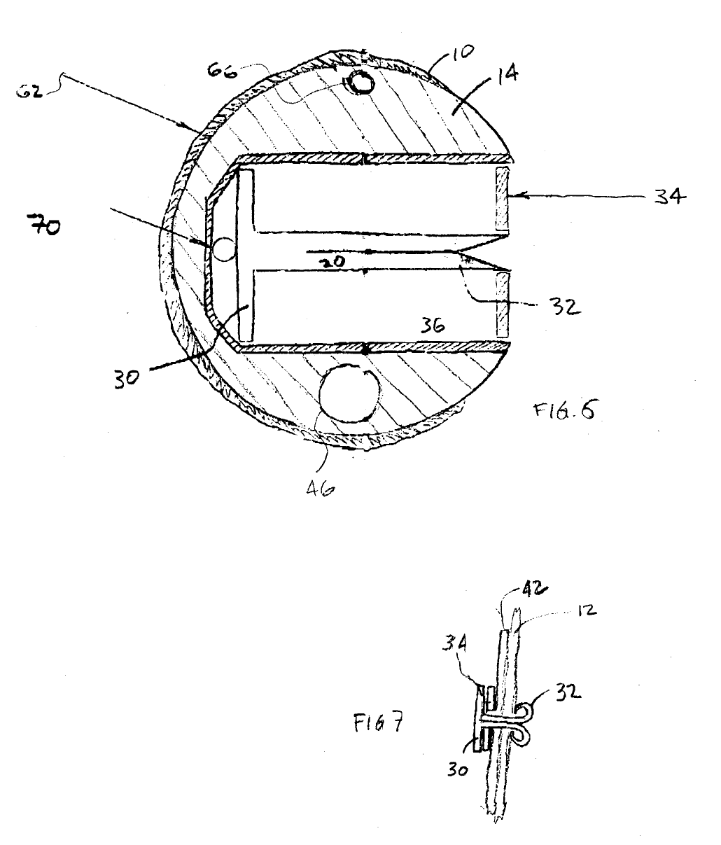

The illustrated rivet 20 has a flat flange 30 that can range in size from 0.5 mm to 2 cm. In FIGS. 1 and 6, the illustrated rivets, seen in a non-deployed state, have straight legs 32 that oppose each other as if they were one leg. Such a rivet can be made of a metal, for example a stainless steel, Elgiloy® alloy (available from Egiloy L. P. of Elgin, Ill., USA), nitinol, a composite material, a synthetic material, or even a bio-absorbable material. The rivet 20 can also take the form of a vascular staple or screw.

The rivet 20 is disposed in a rivet chamber 36 within the catheter 14. In the embodiment of the invention seen in FIGS. 1-4, the flange 30 on the rivet 20 is disposed toward the proximal end of the catheter 14 with the deformable leg or legs 32 extending toward the distal tip of the catheter. This rivet chamber is designed to deploy the rivet out of the end of the catheter.

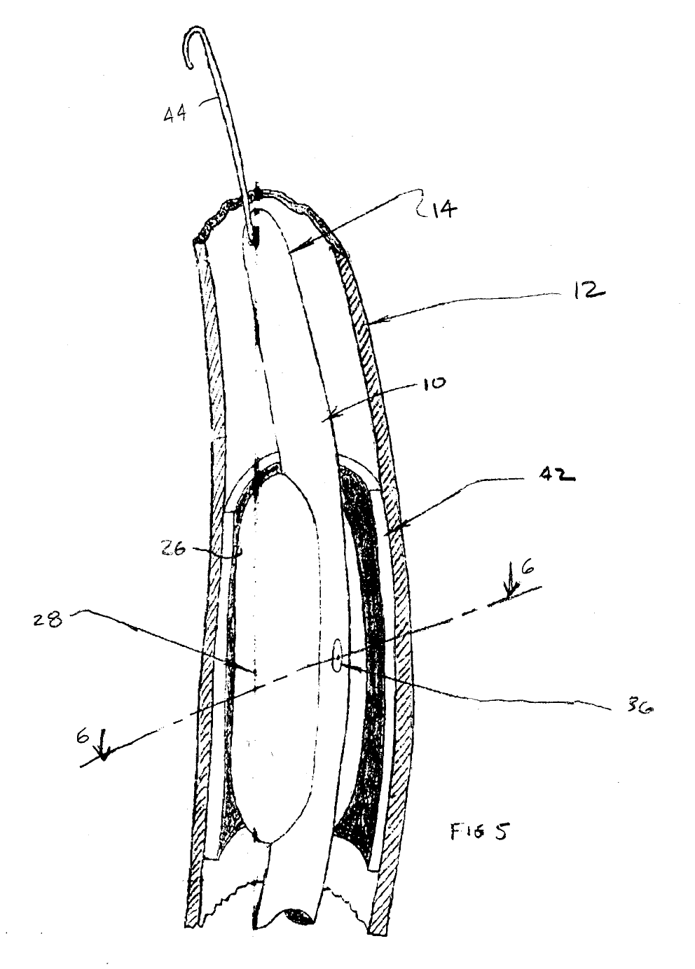

In the alternative embodiment of the invention seen in FIGS. 5 and 6, the rivet chamber 36 is set perpendicularly to the length of the catheter 14. This rivet chamber is designed to deploy the rivet out of the side of the catheter. The device in this embodiment can be guided by a guide wire 44 that passes through a guide wire aperture 46 in the catheter (FIG. 6).

In the illustrated embodiments, a rivet disk 34 is positioned at the outer end of the rivet chamber 36. The rivet disc helps to maintain the rivet 20 in the catheter 14 and retards displacement or false release of the rivet. The illustrated disk is made of a highly elastic material, and provides an interface between rivet and vessel wall to ensure a tight fit. The disk can also serve as the band to help maintain the straight-leg configuration of the rivet prior to deployment.

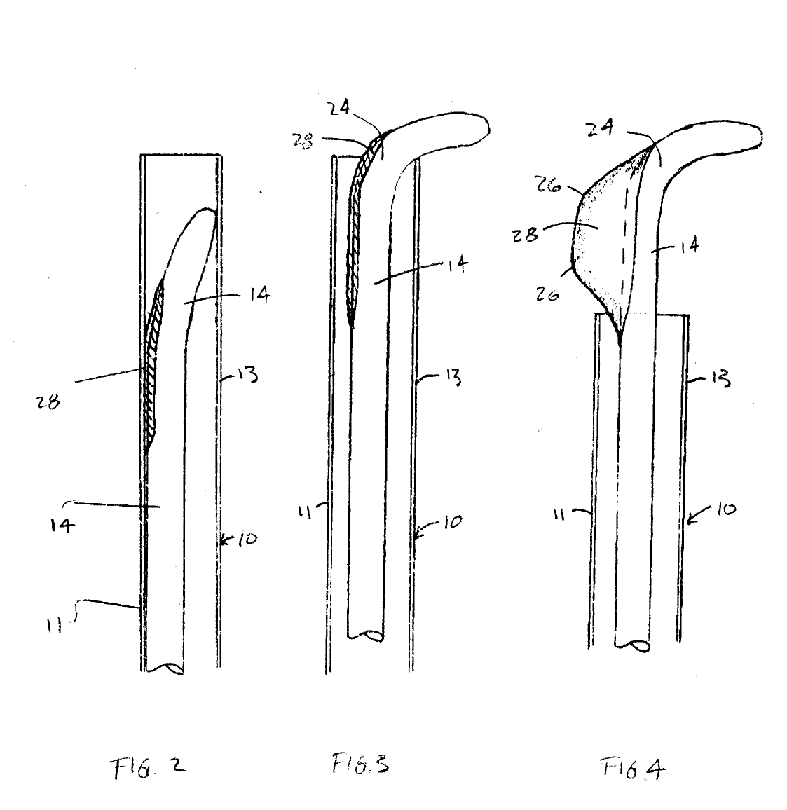

In use, the illustrated deployment catheter 14 advances within the sheath 13 to the intended site of deployment of the rivet 20. The catheter illustrated in FIG. 1 has a pre-formed curve 24 towards its the distal tip. While the distal tip of the catheter is positioned within the sheath, a relatively-straight configuration is imposed, as seen in FIG. 2. When the deployment catheter is advanced beyond the sheath, as seen in FIGS. 3 and 4, the natural curvature of the catheter turns the tip nearly perpendicularly to the wall of the blood vessel. The illustrated curve produces a gradual 90-degree bend once the end of the catheter is pushed out of the sheath.

The illustrated device has contact surfaces 26 on a balloon 28 that is affixed near the distal end of the deployment catheter 14. As seen in FIG. 4, the balloon is preferably eccentric or wedge shaped, and a wider portion may be positioned most distally. The balloon can be inflated so the contact surfaces make rigid and firm contact with the wall of the blood vessel 12, securing the deployment catheter in position. A wedge shape helps exaggerate and maintain the perpendicular position of the tip of the deployment catheter relative to the graft and blood vessel wall. The balloon can be composed of a polyethylene, PET, or other similar material often used in angioplasty balloons.

In the embodiment of the invention seen in FIG. 1, the curved shape of the deployed catheter 14 and the eccentric and preferably wedge shape of the balloon 28 facilitate the 90-degree curve and also anchor the catheter firmly against the graft and the vessel wall.

Instead of a utilizing a preformed curve to facilitate the positioning of the deployment catheter 14 against the wall of the vessel 12, a thread or filament can be fixed to the distal end of the catheter. After the deployment catheter is advanced beyond the sheath, the thread can be pulled to bend the catheter until it has reached the desired shape. To better facilitate such reshaping, the catheter can be configured with a variable thickness or durometer, with one side of the catheter being more rigid and the other side being more flexible. When the thread is pulled, the more-flexible side becomes the inside portion of the curve and the less-flexible side becomes the outer portion of the curve.

For extending the contact surfaces 26 on the balloon 28, the deployment catheter 14 may have a balloon inflation channel 66 on its outer surface or within the channel 16 of the catheter. The illustrated inflation channel is connected to a fluid supply. Adding fluid through the inflation channel inflates the balloon, causing the contact surfaces to extend away from the catheter. The distal tip (or side) of the catheter engages the wall of the blood vessel, temporarily pressing the rivet chamber against the graft and the vessel wall at the location where the rivet is to be deployed.

To install the rivet 20, fluid can be supplied through a fluid channel, which can be the central channel 16 of the deployment catheter 14. The supplied fluid advances to the flange 30 of the rivet and provides the impetus to charge the rivet into the vessel wall. Preferably, the supplied fluid is a gas charge. The gas charge can be held in a small cartridge or connected to a gas supply with a regulator to control a measured discharge of gas. Carbon dioxide gas is ideal for this purpose since it can easily dissolve in blood and can be disposed of in the lungs with normal respiration. Alternatively, helium or others gases could be used.

Alternatively, a reinforced catheter (i.e. braid or spring core), could be used as a mechanical means or pusher to deploy the rivet 20. FIG. 1 shows a pusher 70 that has a dimension and diameter that finds the inner walls of the deployment catheter 12 as a restrictive element, preventing buckling and facilitating the transfer of energy along its length. Thus, a force can be applied along the length of the pusher to cause the legs 32 of the rivet 20 to engage and penetrate the graft and the wall of the blood vessel 12.

The force for the pusher 70 can come from a manual thrust or, alternatively, from a mechanical means of advancing the pusher against the flange 30 of the rivet 20. In order to facilitate the use of a mechanical means to deploy the rivet 20, one clamping device may be placed onto the outer surface of the deployment catheter 14 and a second clamping device can may be placed onto the pusher. A mechanical device can then be used to advance the pusher with a significant force while maintaining the deployment catheter in a stationary fashion.

Once the rivet 20 is deployed, it may be secured in position in several ways. For example, the legs on the vascular rivet may have an initial straight configuration that changes as the rivet pierces through the graft material and aorta, the prongs curling to create a “B” formation much as does a staple. When using a rivet with a heat memory, body temperature warms the legs 32 of the rivet once they are in place, causing them to curl and thereby secure the rivet in place. As seen in FIG. 7, for example, two legs of the rivet have deformed into a “B” configuration, binding a graft 42 into the wall of the blood vessel 12. Alternatively, curved legs made of a resilient material can be bound together in a straight configuration prior to the rivet penetrating the blood vessel, such as by the rivet disk 34 (seen in FIG. 6) or by an elastic or bio-absorbable band. If a bio-absorbable material is used, the bio-absorbable material could be designed to fragment and release the legs of the rivet upon deployment.

The illustrated elastic rivet disc 34, on the other hand, remains in place after the rivet is deployed, and can be used to create a uniform tension between the head of the rivet 20 and the wall of the graft 42. The elasticity of the disc holds the wall of the graft tightly to the aortic wall during geometric changes during the cardiac cycles.

As yet another alternative, the rivet leg 32 can be provided with threads for engaging the blood vessel wall. If the legs 32 of the rivet have threads, the pusher 70 may be used to rotate and drive the leading edge of the threads and thus enable the rivet/screw to advance into the wall of the blood vessel 12.

To prevent an embolization of the rivet 20 in the case of a faulty deployment and failure of the rivet to engage and penetrate the vessel wall, a safety system may be provided. The embodiment illustrated in FIG. 1, for example, includes a tether 22 in the form of a filament or wire that is advanced through a loop on the flange 30 of the rivet. The filament can be made of a polypropylene material such as Prolene Suture (available from Ethicon), or preferably nitinol wire. One end of the filament is fixed externally to a mechanism beyond the sheath 13 and outside of the patient. The other end of the filament is friction fitted by a strong mechanism or by other means to secure the filament to the same said mechanism outside of the sheath. The tether is strung within the inner lumen of the deployment catheter 12. Slack is provided to prevent the tether from restricting the advancement of the rivet as it engages the graft and the vessel wall.

If the rivet 20 is not deployed successfully because (for example) it has improperly or incompletely penetrated the wall of the blood vessel 12, then the tether 22 can be used as a safety cable to hold the rivet and provide a means to retrieve the rivet, if so desired. If, on the other hand, the rivet 20 is deployed successfully, then one end of the tether 22 can be released so the tether can be pulled out of the loop to release the rivet.

If a pusher 70 is used to deploy the rivet 20, then properties of the tether can be incorporated into the pusher. For example, the pusher can be reversibly attached to the flange 30 of the rivet. One way to achieve this is with threads on the pusher that can be disengaged from the rivet by rotating the pusher.

In summary, endovascular devices for treating type 1 leaks from endovascular graft placement can utilize the following principles:

1. A rivet or staple device can be deployed to engage and attach to the wall of a blood vessel, which may also provide a compressive seal to the site of a type 1 leak.

2. A catheter with an extendable mounting surface, such as a surface on a balloon, can be used to secure the device in location.

3. A mechanism can be used for discharging the rivet or staple, such as a charge of carbon dioxide acting on a flange on the rivet, or a mechanical pusher.

4. A tether can be used for possible retrieval of the rivet if it is not successfully deployed.

Many modifications of the invention and the disclosed embodiment that invention will be apparent to those skilled in the art. The scope of protection is defined in the following claims:

What is claimed is:

1) A surgical method comprising the steps of:
providing a surgical device within a patient;
activating the surgical device by discharging a flow of carbon dioxide into a fluid channel in communication with an implement within the patent; and
withdrawing the surgical device from the patient while leaving the implement within the patient.

2) A surgical method as recited in claim 1, in which activating the surgical device secures the implement within the patient.

3) An anchoring device for securing a vascular rivet to the wall of a blood vessel, the device comprising:
a deployment catheter;
a vascular rivet disposed in the catheter; and
a mechanism for deploying the rivet to engage and attach to the wall of the blood vessel.

4) An anchoring device as recited in claim 3, in which the catheter is disposed in a flexible sheath.

5) An anchoring device as recited in claim 3, in which the catheter is disposed in a flexible sheath and the distal end of the deployment catheter has a flexible section that curves when it exits the flexible sheath.

6) An anchoring device as recited in claim 3, in which the catheter is disposed in a flexible sheath and has a thread attached near its distal end.

7) An anchoring device as recited in claim 3, in which a distal end of the deployment catheter has a relatively rigid section opposed to a relatively flexible section.

8) An anchoring device as recited in claim 3, in which the catheter has a mounting surface.

9) An anchoring device as recited in claim 3, in which the device also comprises:
a balloon inflation channel; and
an inflatable balloon in fluid communication with the balloon inflation channel.

10) An anchoring device as recited in claim 3 in which the catheter comprises a wedge-shaped balloon.

11) An anchoring device as recited in claim 3, in which the deploying mechanism comprises a fluid channel in fluid communication with a rivet chamber.

12) An anchoring device as recited in claim 3, in which the deploying mechanism comprises a pusher.

13) An anchoring device as recited in claim 3, in which:
the deploying mechanism comprises a pusher; and
the pusher is releasably connected to the rivet.

14) An anchoring device as recited in claim 3, in which the rivet comprises at least two deformable legs.

15) An anchoring device as recited in claim 3, and further comprising:
a contact surface that is extendable from the catheter to secure the catheter in a location within the blood vessel.

16) An anchoring device as recited in claim 3, in which the rivet has a leg that has a thermal memory and a curved shape at body temperature.

17) An anchoring device as recited in claim 3, in which the rivet has a flange that has a diameter of between 0.1 and 0.2 inches.

18) An anchoring device as recited in claim 3, in which the rivet has a leg that has an end that is disposed in an aperture on a rivet disk.

19) An anchoring device as recited in claim 3, in which the rivet has more than one leg and a binder that releasably holds the legs together.

20) An anchoring device as recited in claim 3, in which the rivet has a threaded leg.

21) An anchoring device is recited in claim 3, in which a filament is connected to the rivet.

22) A vascular rivet with deformable legs that are straight initially and curl when deployed.

23) A vascular rivet as recited in claim 22, in which the rivet is made primarily of one of nitinol, stainless steel, and Elgiloy alloy.

24) A vascular rivet as recited in claim 22, in which the rivet is made primarily of one of a composite material or a synthetic material suitable for clinical application.

25) A vascular rivet for securing a graft to the wall of a blood vessel, the rivet comprising a head and an elastic rivet disc that forms a compressive cushion between the head of rivet and the graft.

26) A vascular rivet as recited in claim 25, in which the rivet is made primarily of one of nitinol, stainless steel, and Elgiloy alloy.

27) A vascular rivet as recited in claim 25, in which the rivet is made primarily of one of a composite material or a synthetic material suitable for clinical application.

28) A method for securing a vascular rivet to the wall of a blood vessel, the method comprising the steps of:
providing a catheter;
disposing a vascular rivet with a flange in the catheter;
positioning the catheter at a location in a blood vessel with the rivet adjacent to a graft;
extending a mounting surface from the catheter to secure the catheter in the location; and
pushing the flange on the rivet to propel a leg of the rivet through the graft and the wall of the blood vessel.

29) A method as recited in claim 28, in which the flange is pushed with a gas.

30) A method as recited in claim 28, in which the flange is pushed with carbon dioxide gas.
